(19) United States
(12) Patent Application Publication
(10) Pub. No.: US 2019/0298584 A1
(43) Pub. Date: Oct. 3, 2019

(54) Moisture Detecting Article and Method of Making Same

(71) Applicant: GUI GLOBAL PRODUCTS, LTD., Houston, TX (US)

(72) Inventor: Walter G. MAYFIELD, Houston, TX (US)

(21) Appl. No.: 16/324,508

(22) PCT Filed: Aug. 11, 2017
(86) PCT No.: PCT/US2017/046592

§ 371 Date: Feb. 8, 2019

Related U.S. Application Data
(60) Provisional application No. 62/374,346, filed on Aug. 12, 2016.

(51) Int. Cl. A61F 13/42; B32B 7/14; B32B 5/02; B32B 5/26; B32B 7/023; B32B 27/12; A61L 15/22; A61L 15/56

(52) U.S. Cl. CPC A61F 13/42; A61F 2013/00846; B32B 5/02; B32B 5/26; B32B 7/023; B32B 27/12; A61L 15/225; A61L 15/56; B32B 2262/0284; B32B 2262/062; B32B 2262/04; B32B 2307/41; B32B 2307/412; B32B 2307/414; B32B 2535/00; B32B 7/14

(57) ABSTRACT

Disclosed is a moisture indicating article having at least 3 layers. The first layer is desirably colored or includes a graphic image and is capable of wicking water. The second layer is a bonding agent. The third layer is also capable of wicking water and is opaque to translucent when dry and becomes very translucent to transparent when wet. The bonding agent allows for the passage of water. When used as part of a dressing, upon getting wet, the color or graphic becomes visible allowing a caretaker to know that it is time to replace the dressing.

### TECHNICAL FIELD

The present invention relates to an article useful for detecting moisture and a method of making it. The present invention particularly relates to such an article that is compatible with medical dressings and wound covers.

### BACKGROUND

Detecting moisture has long been an issue in many areas of human endeavor. For example, modern automobiles are able to detect moisture when it is in the form of rain hitting a windshield.

Another example would be where a catheter is used to access a patient's vascular system for treatment, such as dialysis. During that process, it is known to use an electronic sensor to detect any leakage of blood such as would occur if the catheter were removed or become dislodged.

It would be desirable in the art to be able to determine whether moisture was present in a system simply by looking at it. It would also be desirable if that could be performed without the use of an outside power supply. And it also would be desirable if that could be performed in the presence of an opening in the skin of a human being or animal.

### SUMMARY

In one aspect, the invention is an article having at least 3 layers. The first layer is desirably colored or includes a graphic image and is capable of wicking water. The second layer is a bonding agent. The third layer is also capable of wicking water and is opaque to translucent when dry and becomes very translucent to transparent when wet.

In another aspect, the invention is a method of preparing the article. In this method, the article is prepared by bonding the first layer to the third layer using the second layer bonding agent. It is a characteristic of the bonding agent that it allows the passage of water.

In yet another aspect, the invention is a prophylactic dressing such as that commonly referred to as Band-Aid® brand adhesive bandages.

In still another aspect, the inventions is a prophylactic dressing such as that disclosed in the WO214/1997572 publication claiming priority from U.S. Patent Application No. 61/831,018 and Ser. No. 14/294,945; the contents of which are incorporated herein by reference in their entireties.

### BRIEF DESCRIPTION OF THE DRAWINGS

For a detailed understanding of the present disclosure, references should be made to the following detailed description of the preferred embodiment, taken in conjunction with the accompanying drawings listed below:

FIG. 1 is an illustration of the article of the application;

FIG. 2 is a photograph of an embodiment of the article of the application in a strip form; and

FIG. 3 is photograph of a prophylactic dressing incorporating an article of the application.

### DETAILED DESCRIPTION

In one embodiment, the invention is an article having at least 3 layers. The first layer is desirably colored or includes a graphic image and is capable of wicking water. For the purpose of this application, the term “wicking water” means the ability to allow water to pass through to contact another layer; usually by capillary action.” In some embodiments, the first layer may be in contact with an opening in the skin and should therefore be made for material compatible therewith. In one embodiment the wicking material of the first layer is prepared using polyesters.

Polyesters are homopolymers, copolymers, blends and graft polymers of synthetic long-chain polyesters that contain recurring ester groups in the polymer main chain as an essential constituent. Preferred polyesters are esters of an aromatic dicarboxylic acid with an aliphatic dihydroxy compound, i.e., polyalkylene arylates such as polyethylene terephthalate (PET) or polybutylene terephthalate (PBT).

Such polyalkylene arylates are obtainable by esterifying or transesterifying an aromatic dicarboxylic acid or its esters or ester-forming derivatives with a molar excess of an aliphatic dicarboxy compound and polycondensing the resultant esterification or transesterification product in a known manner.

Useful dicarboxylic acids are 2,6-naphthalenedicarboxylic acid, terephthalic acid and isophthalic acid or mixtures thereof. Up to 30 mol % and preferably not more than 10 mol % of the aromatic dicarboxylic acids may be replaced by aliphatic or cycloaliphatic dicarboxylic acids such as adipic acid, azeleic acid, sebacic acid, dodecanedioic acids and cyclohexanedicarboxylic acids.

Useful aliphatic dihydroxy compounds are diols having 2 to 6 carbon atoms, especially 1,2-ethanediol, 1,3-propanediol, 1,4-butanediol, 1,6-hexanediol, 1,4-hexanediol, 5-methyl-1,5-pentanediol, 1,4-cyclohexanediol, 1,4-cyclohexanedimethanol and neopentyl glycol or mixtures thereof.

One desirable group of polyesters are polyalkylene terephthalates derived from alkanediols having 2 to 10 and preferably 2 to 6 carbon atoms. Of these, particular preference is given to polyethylene terephthalate and polybutylene terephthalate or blends thereof.

Another desirable group of polyesters include those prepared using polyethylene terephthalates and polybutylene terephthalates which contain up to 1% by weight, based on the polyesters, preferably up to 0.75% by weight, of 1,6-hexanediol and/or 5-methyl-1,5-pentanediol as further monomer units.

Such polyalkylene terephthalates contain in the main chain an aromatic ring derived from the aromatic dicarboxylic acid. The aromatic ring may be substituted, for example by halogen such as chlorine and bromine or by C1-C4-alkyl groups such as methyl, ethyl, i-propyl, n-propyl, n-butyl, i-butyl or t-butyl.

Polyester fibers are customarily produced by the melt spinning or the extrusion process, whereafter they are stretched hot. A subsequent heat treatment may be used to render them highly crystalline and shrinkage resistant.

Useful polyester fibers include for example the commercially available Trevira® fibers from Trevira GmbH and Teretal® fibers from Montefibre.

In the case of wovens, the polyester fibers of the fill thread may be identical to or different from the polyester fibers of the warp thread. For instance, the fill may contain PET fibers and the warp PBT fibers, and vice versa.

The second layer is a bonding agent. It is a characteristic of the bonding layer that it allows the passage of water.

In one embodiment, the bonding agent is a layer of adhesive that, while the adhesive does not allow the passage of water, the adhesive is applied in a checker board or mosaic pattern allowing for the passage of water through the areas not covered in adhesive. The adhesive may be applied by spraying, dipping, wiping and any other method known to useful in applying adhesives to a substrate subject to the limitation that the bonding agent must still be able to allow for water passing through to the other layers. The adhesives useful in preparing the articles of the application include but are not limited to silicon and acrylic adhesives.

In another embodiment, the bonding agent is stitching. In this embodiment, stitching is used to hold the layers of the article together.

In yet another embodiment, the bonding agent is a heat activated adhesive. In still another embodiment the bonding agent is a radio wave weldable adhesive.

In a particularly desirable embodiment, the bonding agent is a geometric apertured film in the form of a nonwoven netting fabric which has been coextruded with an adhesive to provide a breathable adhesive film which can also allow water to pass through. An even more desirably embodiment is one where the adhesive is one that can be bonded in multiple ways such as by heat or radio wave welding. Such materials are commercially available from Delstar Technologies and are marketed under the trademark Delnet®-EP.

In still another embodiment, the bonding agent can be hook and eye components or other mechanical means of adhering the two or more other layer of the article together.

The third layer is also capable of wicking water and is opaque to translucent when dry and becomes very translucent to transparent when wet. Exemplary of materials useful as the third layer are any known to those of ordinary skill in the art preparing articles such as the article of this application.

Natural and/or manmade wicking fibers such as cellulosic and/or cellulosic-like material such as cotton and/or Rayon and Rayon derivatives and similar materials such as trilobal Rayon, can be used for the third layer. Other materials which can be used include, but are not limited to materials such as cellulose acetate or other hydrophilic cellulose esters to create, in-situ, a papercloth-like material. Useful Rayon particle size range about 0.2 mm to about 1.0 mm with a denier per filament (dpf) in the range of about 0.8 dpf to about 3 dpf and higher. Useful trilobal Rayon size and weight in denier per filament (dpf) are similar to Rayon and can tend to useful at higher size and dpf ranges. Cotton, bicomponent fibers, polyethylene and polypropylene fibers may also be used alone or in combination with those already listed above.

Nonwoven fibers having both Rayon and polyester are particularly desirable in some applications. For example in some applications a fabric can be prepared as the 3^{rd} layer using from about 10% white Rayon to about 90% white Rayon; and from about 10% white polyester to about 90% white polyester. In other applications a fabric can be prepared as the 3^{rd} layer using from about 20% white Rayon to about 80% white Rayon; and from about 20% white polyester to about 80% white polyester. In still other applications a fabric can be prepared as the 3^{rd} layer using from about 30% white Rayon to about 70% white Rayon; and from about 30% white polyester to about 70% white polyester.

Turning to the third layer, in addition to becoming more transparent when wet, it is important for most embodiments that it also maintain structural integrity and retain its ability to stick to a surface, such as Tegaderm, a trade designation of the 3M company. In applications such as bandages and dressings, these properties are often necessary for a successful commercial product. It would not be acceptable to have a dressing which would fall apart and leave material behind contaminating a wound. While not wishing to be bound by any theory, it is nevertheless believed that at least some polyester functionality may be needed to ensure bonding of Rayon and other such type fibers when wet.

One application for which the articles of the application are particularly suitable is dressings and bandages. For example, a common bandage can be prepared with an article of the application sandwiched between the surface contacting a wound and a water and dirt proof transparent outer layer. When dry, the bandage would appear normal. Should sufficient moisture, either from the wound itself or as contamination seeping into the side the bandage, penetrate the bandage to render the opaque layer transparent, then the color of the first layer or a illustration thereon would become visible indicating that the integrity the bandage to moisture has been breached.

One advantage of the articles of the present application is that they can be reused in some embodiments. The third layer of the article may regain opacity when dried.

While the articles of the present application will have a minimum of 3 layers with the caveat that in some embodiments, the middle layer may be stitching or a mechanical means of holding the other 2 layers together; additional layers may be used with the articles. For example in some embodiments it may be desirable to employ an additional layer of wicking material that is more aesthetically pleasing material adjacent to the first layer. This would be particularly useful when the subject layer would be against skin. Similarly, an additional layer which is configured to prevent the dressing sticking to a wound may be so employed.

As already discussed above, it may be desirable to include an additional layer adjacent to the third layer to prevent contamination of the third layer. For example a clear plastic could be applied to the surface of the third layer to keep it clean and prevent water intrusion from that direction.

In each case a bonding agent would be necessary and would of course sometimes be a bonding agent as already described above. This is especially true for any layer that would be adjacent to the first layer.

In one embodiment, the article of the application may include an additional layer that is adjacent to the third layer and is also wider than the third layer, that is a transparent film coated with an adhesive. This embodiment, which would include bandages, could be prepared in fixed sizes, or it could be prepared and sold in the form of rolls.

In one particularly desirable embodiment, a roll of the article of the application could be used to monitor the integrity of a plastic bag or other form of water exclusion device for use with a limb having a cast or open wound. A process of the application can be performed by first taking a roll of the article of the application and adhering a continuous strip above the cast or wound. Next, a plastic bag could then be placed over the limb to a point above the article of the application and then sealed against water. The person using this method within be able to take a bath or shower and then monitor the article of the application to ensure that no water had penetrated the water exclusion device.

In embodiments where the article of the application is in the form of a roll, the article may employ a tacky adhesive, either with or without a removable protector strip, applied of the surface of the first layer. Also useful with such embodiments, would be a dispenser, especially one which would be useful for cutting the article to desired lengths. In an alternative embodiment, the article may be perforated at selected intervals to allow for the dispensing of standard sizes.

Turning now to FIG. 1, an article of the application is shown (100), wherein the first layer (101) is adhered to the third layer (103) using a bonding agent which is shown as a separate layer (102).

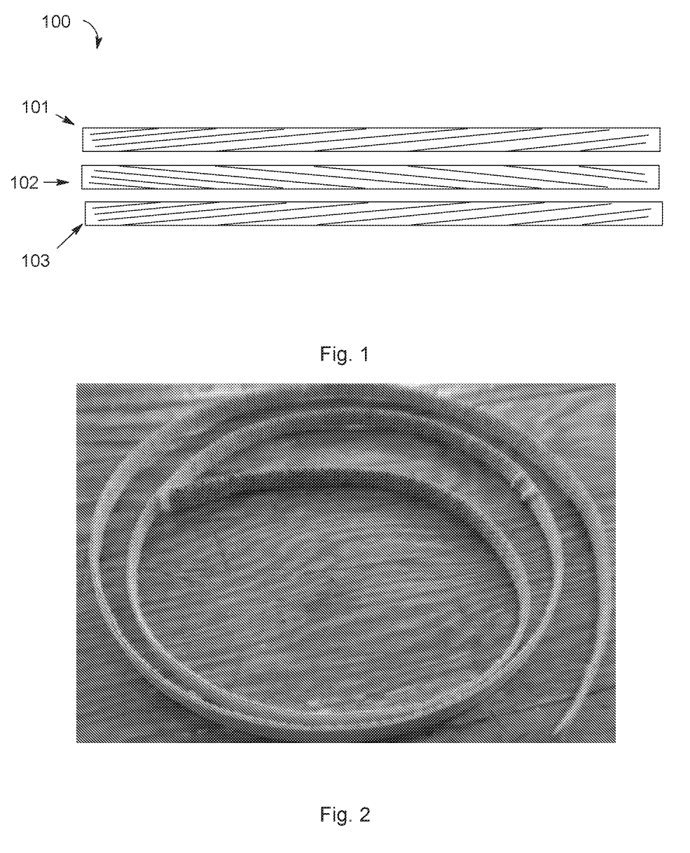

In FIG. 2, a strip of the article of the application is shown with a side view perspective.

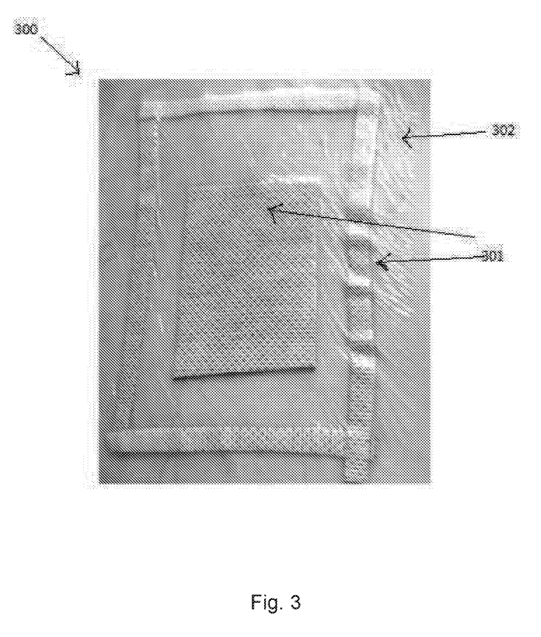

FIG. 3 is an example of a dressing (300). The prophylactic dressing one such as that disclosed in the WO214/1997572 publication of the PCT application claiming priority from U.S. Patent Application No. 61/831,018 and Ser. No. 14/294,945; the contents of which are incorporated herein by reference in their entireties.

The dressing includes a film (302) which has an adhesive to hold the dressing in place. Two articles of the application (301) are shown. When water seeps into the bandage, it is wicked to make the third layer of the article become transparent which in turn allows a colored first layer to be seen.

The articles of the application may be employed in many different end-uses. For example, they may be used to protect scars from sunlight. They may also be employed in dressings having antibiotic properties. In another embodiment, the most or all of the components of the article of the application are elastic allowing for the use of such an article in a dressing that serves to put traction on wound to aid in prevention bleeding or scarring.

The articles of the application may also have other components incorporated therein. For example, in one embodiment, the article of the application may have an additional layer of an expanded foam to allow for longer term wicking of fluids.

1. An article comprising at least 3 layers, wherein:
a first layer is desirably colored or includes a graphic image and is capable of wicking water;
a second layer is a bonding agent; and
a third layer is also capable of wicking water and is opaque to translucent when dry and becomes very translucent to transparent when wet;
wherein the bonding agent functions to adhere the first layer to the third layer.

2. The article of claim 1 wherein the first layer is composed of a polyester.

3. The article of claim 2 wherein the polyester is selected from the group consisting of polyethylene terephthalate, polybutylene terephthalate, and combinations thereof.

4. The article of claim 1 wherein the bonding agent is stitching.

5. The article of claim 1 wherein the bonding agent is an adhesive.

6. The article of claim 5 wherein the bonding agent is an adhesive applied in the form of a checkerboard pattern.

7. The article of claim 5 wherein the bonding agent is an adhesive applied in the form of a mosaic pattern.

8. The article of claim 5 wherein the bonding agent is a silicone adhesive.

9. The article of claim 5 wherein the bonding agent is an acrylic adhesive.

10. The article of claim 1 wherein the third layer is a natural fiber.

11. The article of claim 10 wherein the natural fiber is cotton.

12. The article of claim 1 wherein the third layer is an artificial fiber.

13. The article of claim 12 wherein the artificial fiber is rayon.

14. The article of claim 1 additionally comprising a fourth layer.

15. The article of claim 14 wherein the fourth layer is adjacent to the third layer and wider than the third layer and comprises a transparent film coated with an adhesive.

16. The article of claim 14 wherein the fourth layer is configured to prevent a dressing from sticking to a wound.

17. A method for preparing the article of claim 1 comprising bonding the first layer to the third layer using the second layer bonding agent, wherein the bonding agent allows the passage of water.

18. The method of claim 17 wherein the article is in the form of a roll.

19. A prophylactic dressing prepared using the article of claim 1.
